(19) United States
(12) Patent Application Publication
(10) Pub. No.: US 2005/0214227 A1
(43) Pub. Date: Sep. 29, 2005

(54) Microparticle formulations for sustained-release of bioactive compounds

(75) Inventors: Steven J. Prestrelski, Mountain View, CA (US); Terry L. Burkoth, Palo Alto, CA (US); Gordon M. Saul, Palo Alto, CA (US); Kevin J. Brodbeck, Palo Alto, CA (US)

Correspondence Address: FOLEY AND LARDNER;SUITE 500, 3000 K STREET NW, WASHINGTON, DC, 20007, US

(73) Assignee: Powderject Research Limited

(21) Appl. No.: 10/987,316

(22) Filed: Nov. 15, 2004

Related U.S. Application Data
(63) Continuation of application No. 09/521,139, filed on Mar. 8, 2000, now abandoned.
(60) Provisional application No. 60/123,264, filed on Mar. 8, 1999.

(51) Int. Cl. A61L 9/04; A61K 9/14

(52) U.S. Cl. 424/46

(57) ABSTRACT

A composition is provided for administration to a subject by way of a needleless syringe. The composition is formed from particles having a mean mass aerodynamic diameter of from 1 to 250 microns, and an envelope density of from 0.1 to 25 g/cm³, where the particles include a biologically active agent and a sustained-release material that controls release of the active agent to a subject following administration of the composition thereto. Methods for delivering a biologically active agent to a subject are also provided.

### FIELD OF THE INVENTION

The present invention relates to methods of delivering sustained-release particles containing biological agents into organisms.

### BACKGROUND OF THE INVENTION

The ability to deliver pharmaceuticals through skin surfaces (transdermal or intradermal delivery, collectively “dermal” delivery) provides many advantages over oral or parenteral delivery techniques. In particular, dermal delivery provides a safe, convenient and noninvasive alternative to traditional drug administration systems, conveniently avoiding the major problems associated with oral delivery (e.g., variable rates of absorption and metabolism, food effects, gastrointestinal irritation and/or bitter or unpleasant drug tastes, and inability to deliver large molecules) or other types of parenteral delivery (e.g., needle pain, the risk of introducing infection to treated individuals, the risk of contamination or infection of health care workers caused by accidental needle-sticks and the disposal of used needles). In addition, dermal delivery affords a degree of control over blood concentrations of administered pharmaceuticals.

However, despite its clear advantages, dermal delivery presents a number of its own inherent logistical problems. The passive delivery of drugs through intact skin necessarily entails the transport of molecules through a number of structurally different tissues, including the stratum corneun, the viable epidermis, the papillary dermis, and the capillary walls in order for the drug to gain entry into the blood or lymph system. Transdermal delivery systems must therefore be able to overcome the various resistances presented by each type of tissue.

In light of the above, a number of alternatives to passive transdermal delivery have been developed. These alternatives include the use of skin penetration enhancing agents, or “permeation enhancers,” to increase skin permeability, as well as non-chemical modes such as the use of iontophoresis, electroporation or ultrasound. While these alternatives methods may increase the transdermal delivery of some agents, for other agents these alternatives provide minimal benefit. Furthermore, these alternative techniques often give rise to their own unique side effects, such as skin irritation or sensitization. Thus, the spectrum of pharmaceuticals that can be safely and effectively administered using traditional transdermal delivery methods has remained limited.

More recently, a dermal drug delivery system that entails the use of a needleless syringe to project solid drug-containing particles into and through intact skin has been described. In particular, U.S. Pat. No. 5,630,796 to Bellhouse et al (which is incorporated herein by reference in its entirety) describes a needleless syringe that delivers pharmaceutical particles entrained in a supersonic gas flow. The needleless syringe is used for dermal delivery of drug compounds and compositions, for delivery of genetic material into living cells (e.g., gene therapy) and for the delivery of biopharmaceuticals to skin, muscle, blood or lymph. The needleless syringe can also be used in conjunction with surgery to deliver drugs and biologics to organ surfaces, solid tumors and/or to surgical cavities (e.g., tumor beds or cavities after tumor resection).

Unlike needle or liquid jet injection, the particles of drug delivered by the syringe are sufficiently small that they do not cause tissue distension sufficient to trigger the pain receptors in the skin. Furthermore, by contrast with needles and liquid jet injectors, the syringe eliminates the chance of infection from accidental needlestick injury or splash back of bodily fluids, avoiding the possibility of cross-contamination with blood borne pathogens such as HIV and hepatitis B. Finally, the dry powder compositions used in the syringe are more stable and therefore less expensive to store and distribute and do not require refrigeration or reconstitution before delivery.

The needleless injection technology can be applied to the delivery of biologically active materials to a wide range of organisms ranging from cells to whole animals to man where the biologically active material effects a change within the organism. Generally, the bioactive ingredient used for syringe delivery has been formulated to be readily or instantly available upon delivery to the organism.

In the field of human and veterinary medicine many advantages are offered by sustained-release technology. First, sustained-release of a pharmaceutical agent allows less frequent dosing and thus minimizes handling of animals and repeated treatment of humans. Further, sustained-release treatment results in more efficient drug utilization. Further, less of the compound remains as a residue. Still further, sustained-release technology offers the opportunity to administer and release two or more different drugs, each having a unique release profile, or to release the same drug at different rates or for different durations, by means of a single dosage unit.

However, the physical characteristics of prior sustained-release pharmaceutical compositions have not been selected to meet the demands of administration via a needleless syringe. It is important that particles for delivery via a needleless syringe have a structural integrity and density to survive the action of the gas jet of the syringe and the ballistic impact with skin or mucosal tissue at high velocities. Particles are typically fired at very high velocities from a needleless syringe, including at supersonic velocities such as from Mach 1 to Mach 8.

### SUMMARY OF THE INVENTION

According to the present invention, there is provided a composition suitable for administration to a subject by means of a needleless syringe, which composition comprises particles which have a mean mass aerodynamic diameter of from 1 to 250 microns and an envelope density of from 0.1 to 25 g/cm³, the particles comprising a biologically active agent and a sustained-release material which controls the release of the active agent to the subject following administration.

Particularly preferred are compositions wherein the mean mass aerodynamic diameter of the particles is from about 10 to 100 microns, less than 10% by weight of the particles have a diameter which is 5 microns more or 5 microns less than the mean mass aerodynamic diameter, the envelope density of the particles is from 0.8 to 1.5 g/cm³, the particles exhibit less than 25% reduction in mass mean diameter in a particle attrition test and the particles have an axis ratio of from 3:1 to 1:1. Such compositions are especially able to withstand the forces associated with high velocity particle injection techniques.

The invention also provides:
a hermetically sealed single unit dosage or multidose container adapted for use in a needleless syringe, the said container comprising a composition according to the invention;
a needleless syringe loaded with a composition of the invention;
use of a biologically active agent in the manufacture of a particulate medicament for administration to a subject by means of a needleless syringe, the particles of the medicament having a mean mass aerodynamic diameter of from 0.1 to 250 microns and an envelope density of from 0.1 to 25 g/cm³ and also comprising a sustained-release material which controls the release of the active agent to the subject following administration of the medicament thereto; and
a method for delivering a biologically active agent to an organism, comprising:
   (a) providing particles which have a mean mass aerodynamic diameter of from 0.1 to 250 microns and an envelope density of from 0.1 to 25 g/cm³ and which comprise a biologically active agent and a sustained-release material which controls the release of the active agent to the organism following delivery,
(b) accelerating the particles to a velocity of from 100 to 3000 m/sec, and
   (c) impacting the particles onto a surface of the organism thereby causing the particles to penetrate the surface and enter the organism.

### DETAILED DESCRIPTION OF THE INVENTION

The present invention is concerned with delivering a biologically active agent to an organism by particle injection. The particles comprise a biologically active agent and sustained-release material which controls the release of the active agent to the organism following injection. The particles have a mean mass aerodynamic diameter (MMAD) of from about 0.1 to 250 microns and an envelope density of from 0.1 to 25 g/cm³. The particles have sufficient structural integrity to withstand being fired from a needleless syringe and impacting skin or mucosal tissue at high velocity, typically a supersonic velocity of from Mach 1 to Mach 8.

The particles are typically microcapsules which have encapsulated material, hereinafter referred to as a “biologically active agent”, centrally located within a unique membrane, usually a polymeric membrane. This membrane delays or otherwise controls the agent's release. The membrane may be termed a wall-forming material. Because of their internal structure, permeable microcapsules designed for sustained-release applications can be made which release their agent at a substantially constant rate (zero-order rate of release). Also, impermeable microcapsules can be used for rupture-release applications.

Alternatively, the particles may take the form of microspheres. A microsphere can have the active agent dispersed throughout the particle; that is, the internal structure is a matrix of the active agent and excipient, usually a polymeric excipient known as a “sustained-release material” which affects the release rate. Usually sustained-release microspheres release their agent at a declining rate (first-order). But microspheres can be designed to release agents at a near zero-order rate. Microspheres tend to be more difficult to rupture as compared to microcapsules because their internal structure is stronger. This can be advantageous in view of the rigorous delivery conditions presented by needleless injection. The active material is released from the microparticles by diffusion, leaching or erosion of the matrix (eg. biodegradation) or by a combination of these mechanisms.

The term “microparticle” is synonymous with and includes the terms “microsphere” and “microcapsule”. Preferably the microparticle composition is substantially dry or powder-like, i.e. liquid has not been added to the composition. Some minor amounts of liquid may however remain with the microparticles.

The polymeric matrix material of the microparticles present invention can be composed of a biocompatible and biodegradable polymeric material. The term “biocompatible material” is defined as a polymeric material which is not toxic to an animal and not carcinogenic. The matrix material is preferably biodegradable in the sense that the polymeric material should degrade by bodily processes in vivo to products readily disposable by the body and should not accumulate in the body. On the other hand, where the microparticle is being inserted into a tissue which is naturally shed by the organism (eg. sloughing of the skin), the matrix material need not be biodegradable.

The microparticles of the present invention usually have a spherical shape, although irregularly-shaped microparticles are possible. When viewed under a microscope, therefore, the particles are typically spheroidal but may be elliptical, irregular in shape or toroidal. The microparticles vary in size, ranging in diameter from 0.1 microns to 250 microns, more preferably, from 10 or 20 microns to 75 microns and most preferably from 30 microns to 70 microns.

The term “sustained-release” as used herein encompasses the term “controlled-release” and means that the biologically active agent is released from the microparticle polymeric matrix over an extended period of time so as to give continuing or delayed dosage of the treated organism. The controlled-release period can be from a few hours to 1 to 500 days or longer and preferably is from 3 to 60 days.

The duration of release of the active agent from the microparticle can be adjusted from less than a week to several months or longer by manipulation of various parameters. The amount (level) of biologically active agent released can also be controlled. The parameters include the polymer composition of the controlled-release material, the polymer molecular weight, the polymer:bioactive agent ratio, microparticle diameter and the presence/absence of a release rate modifier in the composition. Other parameters include bound/unbound drug (with respect to a polymer matrix), hydrophobicity of the drug and/or polymer composition and porosity of the polymer matrix.

Mixed populations of different types of microparticles can be combined into single dosage forms and can be co-administered. The same active agent can be incorporated into the different microparticle types that are combined in the final formulation or co-administered. Thus, multiphasic delivery of the same active agent can be achieved.

Alternatively, two or more active agents can be combined in the microparticle composition. The additional bioactive agent(s) can be unencapsulated, encapsulated separately, or co-encapsulated with the first active agent. If separately encapsulated, the matrix may be the same or different for each agent at predetermined release rates with the release of each drag being at a different rate and duration by design. As above, any of the parameters may be varied to produce unique microparticles having specific release characteristics for the given encapsulated agent. Thus, distinct multiphasic release patterns can be obtained for each of the agents encapsulated in mixed microparticle populations for single or co-administration. The invention thus provides a wide range of possible in vivo release profiles.

The term “administer” or “deliver” is intended to refer to any delivery method using needleless syringes or the like means of accelerating the microparticle composition onto the tissue or skin or wall of the organism thereby causing the microparticles to penetrate the surface and become available to the organism. When administration is for the purpose of treatment, administration may be either for prophylactic or therapeutic purpose. When provided prophylactically, the bioactive agent is provided in advance of any symptom. The prophylactic administration of the bioactive agent serves to prevent or attenuate any subsequent symptom. When provided therapeutically the bioactive agent is provided at (or shortly after) the onset of a symptom. The therapeutic administration of the bioactive agent serves to attenuate any actual symptom.

The terms “needleless syringe,” and “needleless syringe device,” as used herein, expressly refer to a particle delivery system that can be used to deliver the microparticles into and/or across tissue. The particles have an average size ranging from about 0.1 to 250 microns. Particles larger than about 250 microns can also be delivered from these devices, with the upper limitation being the point at which the size of the particles would cause untoward pain and/or damage to the target tissue. The microparticles are delivered by the needleless syringe at high velocity, preferably at velocities of at least about 100 meters/second or greater, more preferably at velocities of about 250 deters/second or greater and most preferably at velocities of at least about 300 meters/second or greater. Such needleless syringe devices are described in U.S. Pat. No. 5,630,796 to Bellhouse et al, incorporated herein by reference in its entirety, and have since been described in International Publication Nos. WO 96/04947, WO 96/12513, and WO 96/20022, all of which publications are also incorporated herein by reference in their entirety. Since the term only relates to devices which are suitable for delivery of particulate materials, devices such as liquid-jet injectors are expressly excluded from the definition of a “needleless syringe.”

The term “organism” refers to eukaryotic cells whether in vivo or in vitro, eukaryotic tissues whether in vivo or in vitro, and animals, individuals, patients and subjects as defined herein.

The terms “animal”, “individual”, “patient” and “subject” are used interchangeably herein to refer to a subset of organisms which include any member of the subphylum cordata, including, without limitation, humans and other primates, including nonhuman primates such as chimpanzees and other apes and monkey species; farm animals such as bovine animals, for example cattle; ovine animals, for example sheep; porcine, for example pigs; rabbit, goats and horses; domestic mammals such as dogs and cats; wild animals; laboratory animals including rodents such as mice, rats and guinea pigs; birds, including domestic, wild and game birds such as chickens, turkeys and other gallinaceous birds, ducks, geese; and the like. The terms do not denote a particular age. Thus, both adult and newborn individuals are intended to be covered.

The term “tissue” refers to the soft tissues of an animal, patient, subject etc as defined herein, which term includes, but is not limited to, skin, mucosal tissue (eg. buccal, conjunctival, gums), vaginal and the like. Bone may however be treated too by the particles of the invention, for example bone fractures.

The methods of this invention include treating a disease state in an animal by administering the microparticle composition described herein by a needleless syringe to a subject in need of such treatment. As used herein, the term “treatment” or “treating” includes any of the following: the prevention of infection or reinfection; the reduction or elimination of symptoms; and the reduction or complete elimination of a pathogen. Treatment may be effected prophylactically (prior to infection) or therapeutically (following infection). The methods of this invention also include effecting a change in an organism by administering the microparticle compositions.

A “therapeutically effective amount” is defined very broadly as that amount needed to give the desired biologic or pharmacologic effect. This amount will vary with the relative activity of the agent to be delivered and can be readily determined through clinical testing based on known activities of the compound being delivered. The “Physicians Desk Reference” and “Goodman and Gilman's The Pharmacological Basis of Therapeutics” are useful for the purpose of determined the amount needed in the case of known pharmaceutical agents. The amount of agent administered depends on the organism( eg. animal species), agent, route of administration, length of time of treatment and, in the case of animals, the weight, age and health of the animal. One skilled in the art is well aware of the dosages required to treat a particular animal with an agent. Commonly, the agents are administered in milligram amounts.

The term “sustained-release material” includes both wall-forming materials for use in microcapsules and polymeric materials for use in microspheres. The sustained-release material should be biologically acceptable. A “biologically acceptable” material is one which can be present in a biological environment without causing unwanted deleterious effects.

Suitable wall-forming materials for use in microcapsules include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitrites), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. See for example International Patent Application No. PCT/GB00/00349 which is incorporated herein by reference. The polymeric materials may be cross-linked.

These materials may be used alone, as physical mixtures blends) or as copolymers (which may be block copolymers). A preferred group of wall-forming materials includes biodegradable polymers such as poly(lactide), poly(glycolide), poly(caprolactone), poly(hydroxybutyrate), and copolymers thereof including but not limited to poly(lactide-co-glycolide), poly(lactide-co-caprolactone) and the like. Again, these polymers may be cross-linked. The copolymers may be block, random or regular copolymers.

The molecular weight of the polymeric material can be of some importance The molecular weight should be high enough so that it forms satisfactory polymer coatings, i.e., the polymer should be a good film former. Usually, a satisfactory molecular weight is in the range of 5,000 to 500,000 daltons, more preferably in the range of 10,000 to 500,000 daltons although it can be higher. The biodegradable polymers mentioned above generally have molecular weights of from 30,000 to 50,000 daltons, up to about 120,000 daltons such as from 80,000 to 100,000 daltons. Dextrans may have an upper molecule weight of about 500,000. However, since the properties of the film and the processing conditions are also partially dependent on the particular polymeric material being used, it is very difficult to specify an appropriate molecular weight range for all polymers.

The molecular weight of a polymer is also important from the point of view that molecular weight influences the biodegradation rate of the polymer. For a diffusional mechanism of drug release, the polymer should remain intact until all of the drug is released from the microparticles and then degrade. The drug can also be released from the microparticles as the polymeric excipient bioerodes. By an appropriate selection of polymeric materials a microparticle formulation can be made such that the resulting microparticles exhibit both diffusional release and biodegradation release properties. This is useful in affording multiphasic release patterns. Bimodal release of active agent can be achieved by providing two types of particles having polymeric matrix materials of different molecular weight.

Inclusion of a hydrophobic solvent as an additive to the polymer can prolong and control drug release within a designated therapeutic window. Thus, weak solvents, with a low affinity for water, such as N-methyl-2-pyrrolidone or triacetin, can be added to a polymer material such as poly(lactide-co-glycolide) to dissolve partially the matrix and thereby block the pores of the matrix from direct diffusion of incoming water. In this way, release of the biologically active agent can be governed in the initial stages after injection by the affinity of the solvent employed and water.

Any biologically acceptable biodegradable matrix polymer may be used in the generation of microspheres. Suitable examples of polymeric matrix materials include poly(glycolic acid), polyd,1-lactic acid, poly-1-lactic acid, copolymers of the foregoing, poly(aliphatic carboxylic acids), polycaprolactone, polydioxonene, poly(ortho carbonates), poly(acetals), poly(lactic acid-caprolactone), polyorthoesters, poly(glycolic acid-caprolactone), polydioxonene, polyanhydrides, polyposphazopnes, polyethyleneglycol, poly(hydroxy-butyric acid), poly(orthoesters) and natural polymers including, polyamino acids, albumin, casein, and some waxes, such as, glycerol mono- and distearate, and the like. The polymer may be cross-linked.

Various commercially available poly (lactide-co-glycolide) materials (PLGA) may be used in the method of the present invention. For example, poly (d,1-lactic-co-glycolic acid) is commercially available from Arkermes (formerly Medisorb) Technologies International LAP. (Cincinnati, Ohio). A suitable product commercially available from Medisorb is a 50:50 poly (d,1) lactic co-glycolic acid known as MEDISORB.RTM. 5050 DL. This product has a mole percent composition of 50% lactide and 50% glycolide. Other suitable commercially available products are MEISORB.RTM. 65:35 DL, 75:25 DL, 85:15 DL and poly(d,1-lactic acid) (d,1.PLA). Poly(lactide-co-glycolides) are also commercially available from Boehringer Ingelheim (Germany) under its Resomer mark, e.g., PLGA 50:50 (Resomer RG 502), PLGA 75:25 (Pesomer RG 752) and d,1-PLA (Resomer RG 206), and from Birmingham Polymers (Birmingham, Ala.). These copolymers are available in a wide range of molecular weights and ratios of lactic acid to glycolic acid. A preferred polymer for use in the practice of this invention is poly(d,1-lactide-co-glycolide). It is preferred that the molar ratio of lactide to glycolide in such a copolymer be in the range of from about 95:5 to about 50:50.

The molecular weight of the polymeric material can be of some importance. The molecular weight should be high enough so that it forms satisfactory polymer coatings, i.e., the polymer should be a good film former. Usually, a satisfactory molecular weight is in the range of 20,000 to 2,000,000 daltons with, in some instances, physical cross-linking, more preferably in the range of 10,000 to 500,000 daltons although it can be higher. The biodegradable polymers mentioned above generally have molecular weights of from 50,000 to 50,000 daltons, up to about 120,000 daltons such as from 80,000 to 100,000 daltons. Dexam may have an upper molecule weight of about 2,000,000 to 3,000,000. However, since the properties of the film End the processing conditions are also partially dependent on the particular polymeric material being used it is very difficult to specify an appropriate molecular weight range for all polymers.

The molecular weight of a polymer is also important from the point of view that molecular weight influences the biodegradation rate of the polymer. For a diffusional mechanism of drug release, the polymer should remain intact until all of the drug is released from the microparticles and then degrade. The drug can also be released from the microparticles as the polymeric excipient bioerodes. By an appropriate selection of polymeric materials a microparticle formulation can be made such that the resulting microparticles exhibit both diffusional release and biodegradation release properties. This is useful in affording multiphasic release patterns. Bimodal release of active agent can be achieved by providing two types of particles having polymeric matrix materials of different molecular weight.

Inclusion of a hydrophobic solvent as an additive to the polymer matrix can prolong and control drug release within a designated therapeutic window. Thus, weak solvents, with a low affinity for water, such as N-methyl-2-pyrrolidone or triacetin, can be added to a polymer matrix such as poly(lactide-co-glycolide) to dissolve partially the matrix and thereby block the pores of the matrix from direct diffusion of incoming water. In this way, release of the biologically active agent can be governed in the initial stages after injection by the affinity of the solvent employed and water.

In one method, the microsphere includes an inert particle core. The inert particle core includes, but is not limited to, metals such as gold, tungsten, or platinum, metal compounds such as ferrite, and organic polymers such as polystyrene or latex. Such core material are well known in the art. Typically, the microparticle will not comprise an inert particle core.

A “penetration enhancer” or “permeation enhancer” as used herein is a material which increases the permeability of skin or other material to a biologically active agent remaining or present on the surface of the skin, e.g., so as to increase the rate at which the drug permeates through the skin and enters the bloodstream. The enhanced permeation effected through the use of such enhancers can be observed by measuring the rate of diffusion of drug through animal or human skin using a diffusion cell apparatus well known in the art. Penetration enhancers may be used to facilitate transport of any residual biologically active agent left on the skin at the delivery site by the methods of this invention. An “effective” amount of a permeation enhancer as used herein means an amount that will provide the desired increase in amount of drug delivered by the method of this invention.

A “biologically-active agent” includes any compound or composition of matter which, when administered to an organism induces a desired pharmacologic and/or physiologic effect by local and/or systemic action. The term therefore encompasses those compounds or chemicals traditionally regarded as drugs, biopharmaceuticals (including molecules such as peptides, proteins, nucleic acids), vaccines and gene therapies (e.g., gene constructs).

Biologically-active agents useful in the composition of this invention include drugs acting at synaptic and neuroeffector junctional sites (cholinergic agonists, anticholinesterase agents, atropine, scopolamine, and related antimuscarinic drugs, catecholamines and sympathomimetic drugs, and adrenergic receptor antagonists); drugs acting on the central nervous systems; drugs affecting renal function and electrolyte metabolism; cardiovascular drugs; drugs affecting gastrointestinal function; chemotherapy of neoplastic diseases; drugs acting on the blood and the blood-forming organs; and hormones and hormone antagonists. Thus, the agents useful in the composition include, but are not limited to anti-infectives such as antibiotics and antiviral agents; analgesics and analgesic combinations; local and general anesthetics; anorexics; anti-arthritics; anti-asthmatic agents; anti-convulsants; antidepressants; antihistamines; anti-inflammatory agents; anti-nauseants; anti-migraine agents; anti-neoplastics; anti-pruritics; anti-psychotics; antipyretics; antispasmodics; cardiovascular preparations (including calcium channel blockers, beta-blockers, beta-agonists and anti-arrythmics); anti-hypertensives; diuretics; vasodilators; central nervous system stimulants; cough and cold preparations; decongestants; diagnostics; hormones; bone growth stimulants and bone resorption inhibitors; immunosuppressives; muscle relaxants; psychostimulants; sedatives; and tranquilizers.

Specific examples of drugs useful in this invention include angiotensin converting enzyme (ACE) inhibitors, β-lactam antibiotics and γ-aminobutyric acid (GABA)-like compounds. Representative ACE inhibitors are discussed in Goodman and Gilman Eighth Edition at pp. 757-762, which is incorporated herein by reference. These include quinapril, ramipril, captopril, benzepril, fosopril, lisinopril, enalapril, and the like and the respective pharmaceutically acceptable salts thereof. Beta-lactam antibiotics are those characterized generally by the presence of a beta-lac ring in the structure of the antibiotic substance and are discussed in Goodman and Gilman, Eighth Edition at pp. 1065 to 1097, which is incorporated herein by reference. These include penicillin and its derivatives such as amoxicillin and cephalosporins. GABA-like compounds may also be found in Goodman and Gilman, Other compounds include calcium channel blockers (e.g., verapamil, nifelipine, nicardipine, nimodipine and diltiazem); bronchodilators such as theophylline; appetite suppressants, such as phenylpropanolamine hydrochloride; antitussives, such as dextromethorphan and its hydrobromide, noscapine, carbetapentane citrate, and chlophedianol hydrochloride; antihistamines, such as terfenadine, phenidamine tartrate, pyrilamine maleate, doxylamine succinate, and phenyltoloxamine citrate; decongestants, such as phenylephrine hydrochloride, phenylpropanolamine hydrochloride, pseudoephedrine hydrochloride, chlorpheniramine hydrochloride, pseudo ephedrine hydrochloride, chlorpheniramine maleate, ephedrine, phenylephrine, chlorpheniramine, pyrilamine, phenylpropanolamine, dexchlorpheniramine, phenyltoxamine, phenindamine, oxymetazoline, methscopalunine, pseudoephedrine, brompheniramine, carbinoxamine and their pharmaceutically acceptable salts such as the hydrochloride, maleate, tannate and the like, β-adrenergic receptor antagonists (such as propanolol, nadalol, timolol, pindolol, labetalol, metoprolol, atenolol, esniolol, and acebutolol); narcotic analgesics such as morphine; central nervous system (CNS) stimulants such as methylphenidate hydrochloride; antipsychotics or psychotropics such as phenothiazine, trycyclic antidepressants and MAO inhibitors; benzadiazepines such as alprazolam, diazepam; and the like; and certain non steroidal antinflammatory drugs (NSAIDs), (e.g., salicylates, pyrazolons, indomethecin, sulindac, the fenamates, tolmetin, propionic acid derivatives) such as salicylic acid, aspirin, methyl salicylate, diflunisal, phenylbutazone, indomethacin, oxyphenbutazone, apazone, mefenamic acid, meclofenamate sodium, ibuprofen, naproxen, naproxen sodium, fenoprofen, ketoprofen, flurbiprofen, piroxicam, diclofenac, etodolac, ketorolac, aceclofenac, naburnetone, and the like.

Additional examples of drugs that may be employed in the present compositions and methods include analgesics such as acetaminophen and the like; anesthetics such as lidocaine, xylocaine, and the like; anorexics such as dexedrine, phendimetrazine tartrate, and the like; antiarthritics such as methylprednisolone, ibuprofen, and the like; anti-asthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erytromycin, clindamycin, isoniazid, rifampin, and the like; antifungals such as amphotericin B, nystatin, ketoconazole, and the like; antivirals such as acyclovir, amantadine, and the like; anti-cancer agents such as cyclophosphamide, methotrexate, etretinate, and the like; anticoagulants such as heparin, warfarin, and the like; anticonvulsants such as phenytoin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins, and minerals such as essential amino acids, calcium, iron, potassium, zinc, vitamin B₁₂, and the like; cardiovascular agents such as prazosin HCl, nitroglycerin, propranolol HCl, hydralazine HCl, verapamil HCl, and the like; prostaglandins; carbohydrates; fats; narcotics such as morphine, codeine, and the like; psychotherapeutics; anti-malarials; L-dopa, diuretics such as furosemide, spironolactone, and the like; antiulcer drugs such as ranitidine HCl; cimetidine HCl, and the like.

Biologically active agents that can also be incorporated in the microparticles of the present invention include proteins, peptides, and fragments thereof (whether naturally occurring, chemically synthesized or recombinantly produced) such as LHRH and analogs thereof, somatostatin, calcitonin, parathyroid hormone, growth hormone, growth hormone releasing factor, GnRH (gonadotrophin releasing hormone), angiotensin, FSH, EGF, vasopressin, ACTH human serum albumin, gamma globulin, interleukins, interferons, colony stimulating factors, tumor necrosis factor and the like; enzymes such as lactase, pancrelipase, succinic acid dehydrogenase, and the like.

Another biologically active agent useful in the composition and method of this invention is an antigen, i.e., molecule which contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. The antigen can be derived from any known virus, bacterium, parasite, plants, protozoans, or fungus, and can be a whole organism or immunogenic parts thereof, e.g., cell wall components. An antigen can also be derived from a tumor. An oligonucleotide or polynucleotide which expresses an antigen, such as in DNA immunization applications, is also included in the definition of antigen. Synthetic antigens are also included in the definition of antigen, for example, haptens, polyepitopes, flanking epitopes, and other recombinant or recombinant or synthetically derived antigens (Bergmann et al. (1993) Eur. J. Immunol. 23:2777-2781; Bergmann et al. (1996) J. Immunol. 157:3242-3249; Suhrbier, A. (1997) Immunol. And Cell Biol. 75:402-408; Gardner et al. (1998) 12^{th} World AIDS Conference, Geneva, Switzerland (Jun. 28-Jul. 3, 1998).

When an antigen is associated with a sustained-release material in accordance with the invention, it can be viewed as a “vaccine composition” and as such includes any pharmaceutical composition which contains an antigen and which can be used to prevent or treat a disease or condition in a subject. The term encompasses both subunit vaccines, i.e., vaccine compositions containing antigens which are separate and discrete from a whole organism with which the antigen is associated in nature, as well as compositions containing whole killed, attenuated or inactivated bacteria, viruses, parasites or other microbes. The vaccine can also comprise a cytokine that may further improve the effectiveness of the vaccine.

Viral vaccine compositions used herein include, but are not limited to, those containing, or derived from, members of the families Picornaviridae (e.g., polioviruses, etc.); Caliciviridae; Togaviridae (e,g., rubella virus, dengue virus, etc.); Flaviviridae; Coronaviridae; Reoviridae; Birnavirndae; Rhabodoviridae (e.g., rabies virus, measels virus, respiratory syncytial virus, etc.); Orthomyxoviridae (e.g., influenza virus types A, B and C, etc,); Bunyaviridae; Arenaviridae; Retroviradae (e.g., HTLV-I; HTLV-II; HIV-1; and HIV-2); simian immunodeficiency virus (SIV) among others. Additionally, viral antigens may be derived from a papilloma virus (e.g., HPV); a herpes virus, i.e. herpes simplex 1 and 2; a hepatitis virus, e.g., hepatitis A virus (HAV), hepatitis B virus (HBV), hepatitis C virus (HCV), the delta hepatitis D virus (HDV), hepatitis E virus (HEV) and hepatitis G virus (HGV) and the tick-borne encephalitis viruses; smallpox, parainfluenza, varicella-zoster, cytomeglavirus, Epstein-Barr, rotavirus, rhinovirus, adenovirus, papillomavirus, poliovirus, mumps, rubella, coxsackieviruses, equine encephalitis, Japanese encephalitis, yellow fever, Rift Valley fever, lymphocytic choriomeningitis, and the like. See e.g., Virology, 3^{rd} Edition (W. K. Joklik ed. 1988); Fundamental Virology, 2^{nd} Edition (B. N. Fields and D. M. Knipe, eds. 1991), for a description of these and other viruses.

Bacterial vaccine compositions used herein include, but are not limited to, those containing or derived from organisms that cause diphtheria, cholera, tuberculosis, tetanus, pertussis, meningitis, and other pathogenic states, including Meningococcus A, B and C, Hemophilus influenza type B (HIB), and Helicobacter pylori; Streptococcus pneumoniae, Staphylococcus aureus, Streptococcus pyrogenes, Corynebacterium diphtheriae, Listeria monocylogenes, Bacillus anathracis, Clostridium tetani, Clostridium botulinum, Clostridium perfringens, Neisseria meningtidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycrobacterium tuberculosis, Mycobacterium leprae, Treponema pallidum, Leptspirosis interrogans, Borrelia burgdoferi, Campylobacter jejuni, and the like.

Examples of anti-parasitic vaccine compositions include those derived from organisms causing malaria and Lyme disease. Antigens of such fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma bruce, Entamoeba histolytica, Toxoplasma gondii, Thichomonas vaginalis, Schistosoma mansoni, and the like.

Other antigens include allergens such as cat dander, birch pollen, house dust mite, grass pollen, and the like.

Suitable nucleotide sequences for use in the present invention include any therapeutically relevant nucleic acid molecules (polymeric forms of two or more nucleotides, either ribonucleotides (RNA) or deoxyribonucleotides (DNA)) including double- and single-stranded molecules and supercoiled or condensed molecules, gene constructs, expression vectors, plasmids, antisense molecules and the like, Thus, the present invention can be used to deliver one or more genes encoding a protein defective or missing from a target cell genome or one or more genes that encode a non-native protein having a desired biological or therapeutic effect (e.g., an antiviral function). The invention can also be used to deliver a nucleotide sequence capable of providing immunity, for example an immunogenic sequence that serves to elicit a humoral and/or cellular response in a subject, or a sequence that corresponds to a molecule having an antisense or ribozyme function.

Suitable genes which can be delivered include those used for the treatment of inflammatory diseases, autoimmune, chronic and infectious diseases, including such disorders as AIDS, cancer, neurological diseases, cardiovascular disease, hypercholestemia; various blood disorders including various anemias, thalassmaia and hemophilia; genetic defects such as cystic fibrosis, Gaucher's Disease, adenosine deaminase (ADA) deficiency, emphysema, etc. A number of antisense oligonucleotides (e.g., short oligonucleotides complementary to sequences around the translational initiation site (AUG codon) of an mRNA) that are useful in antisense therapy for cancer and for viral diseases have been described in the art. See, e.g., Han et al. 1991) Proc. Natl. Acad Sci. USA 88:4313; Uhlmann et al. (1990) Chem. Rev. 20:543; Helene et al. (1990) Biochim. Biophys. Acta. 10499; Agarwal et al. (1988) Proc, Natl. Acad Scan USA 85;7079; and Heikkila et al. (1987) Nature 38:445. A number of ribozymes suitable for use herein have also been described. See, e.g., Chec et al. (1992) J. Boil. Chem. 267:17479 and U.S. Pat. No. 5,225,347 to Goldberg et al.

For example, in methods for the treatment of solid tumors, genes encoding toxic peptides (i.e., chemotherapeutic agents such as ricin, diphtheria toxin and cobra venom factor), tumor suppressor genes such as p53, genes coding for mRNA sequences which are antisense to transforming oncogenes, antineoplastic peptides such as tumor necrosis factor (TNF) and other cytokines, or transdominant negative mutants of transforming oncogenes, can be delivered for expression at or near the tumor site.

Similarly, genes coding for peptides known to display antiviral and/or antibacterial activity, or stimulate the host's immune system, can also be administered. Thus, genes encoding many of the various cytokines (or functional fragments thereof), such as the interleukins, interferons and colony stimulating factors, will find use with the instant invention. The gene sequences for a number of these substances are known.

For the treatment of genetic disorders, functional genes corresponding to genes known to be deficient in the particular disorder can be administered to the subject. The instant invention will also find use in antisense therapy, e.g., for the delivery of oligonucleotides able to hybridize to specific complementary sequences thereby inhibiting the transcription and/or translation of these sequences. Thus DNA or RNA coding for proteins necessary for the progress of a particular disease can be targeted, thereby disrupting the disease process. Antisense therapy, and numerous oligonucleotides which are capable of binding specifically and, predictably to certain nucleic acid target sequences in order to inhibit or modulate the expression of disease-causing genes are known and readily available to the skilled practitioner.

Uhlmann et al. (1990) Chem Rev. 90:543, Neckers et al (1992) Crit. Rev. Oncogenesis 3:175; Simons et al. (1992) Nature 359:67; Bayever et al. (1992) Antisense Res. Dev. 2:109; Whitesell et al. (1991) Antisense Res. Dev. 1:343; Cook et al. (1991) Anti-cancer Drug Design 6:585; Eguchi et al. (1991) Annu. Rev. Biochem. 60:631. Accordingly, antisense oligonucleotides capable of selectively binding to target sequences in host cells are provided herein for use in antisense therapeutics.

Preferably, when the biologically active agent is a nucleic acid, the nucleic acid is not linked or co-existing with other molecules or promoting materials that facilitate the uptake and integration of the genetic material into living cells. Such promoting materials are selected from the group consisting of glycoprotein, lipoprotein, nucleoproteins and peptides, hormones, antibodies, growth factors, nucleic acid binding factors, proteinaceous cellular ligands, glycolipids, peptidoglycans, lectins, fatty acids, phospholipids, glycolipids, triglycerides, steroid hormones, cholesterol, single-stranded or double stranded DNA and intercalating agents.

The composition of the invention may also include “release rate modifiers” which are pharmaceutically acceptable excipients, other than the biologically active agent and the controlled-release material, which modify the release of the biologically active agent from the microparticle. Such release rate modifiers include but are not limited to zinc and various pharmaceutically acceptable surfactants.

The composition of the invention may also include pharmaceutically acceptable excipients in addition to the rate controlling materials such as a binder, carrier, stabilizer, glidant, antioxidant, pH adjuster, buffer, anti-irritant, and the like. Such an “excipient” generally refers to a substantially inert material that is nontoxic and does not interact with other components of the composition in a deleterious manner.

Examples of suitable carriers that also act as stabilizers for peptide include pharmaceutical grades of dextrose, sucrose, lactose, trehalose, mannitol, sorbitol, inositol, dextran, and the like. Other carriers include starch, cellulose, sodium or calcium phosphates, calcium sulfate, citric acid, tartaric acid, glycine, high molecular weight polyethylene glycols (PEG), and combinations thereof. It may also be useful to employ a charged lipid and/or detergent. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, e.g., TWEEN® surfactants (Atlas Chemical Industries, Wilmigton, Del.), polyoxyethylene ethers, e.g. Brij, pharmaceutically acceptable fatty acid esters, e.g., lauryl sulfate and salts thereof (SDS), and like materials.

The microparticles can comprise from about 1 to about 99 weight percent of the biologically active agent, for example from about 10 to about 80 weight percent of the biologically active agent. The biologically active agent can therefore be present in amounts of from 2.0 to 70 weight percent such as from 25 to 60 weight percent or 25 to 50 weight percent. The actual amount depends on a number of factors include the nature of the biologically active agent, the dose desired and other variables readily appreciated by those skilled in the art.

Microparticles of the present invention can be designed to provide unique release characteristics. The parameters which are varied to produce the characteristics include the polymer composition, polymer molecular weight, polymer:biologically active agent ratio, and microparticle diameter. Heterogeneous compositions of uniquely designed microparticles are also encompassed in the present invention. Any permutation of the parameters designed to produce a desired release profile is within the scope of the invention. For example, all microparticles in the final formulation may be of approximately similar size but have different drug loadings (wt %). Alternatively, the same weight percent may be found in all of the microparticles but the sizes may differ. Further, heterogeneous populations of microparticles may include subpoulations formed with different polymer matrices, etc.

A variety of methods are known by which compounds can be encapsulated in microparticles. In these methods, the material to be encapsulated (drugs or other active agents) is generally dispersed or emulsified, using stirrers, agitators, or other dynamic mixing techniques, in a solvent containing a wall-forming material. Solvent is then removed from the microparticles, and thereafter the microparticle product is obtained. Typically, the particles of the invention are provided as a free-flowing powder.

An example of a conventional microencapsulation process for pharmaceutical preparations is shown in U.S. Pat. No. 3,737,337, incorporated herein by reference in its entirety. The substances to be encapsulated or embedded are dissolved or dispersed in the organic solution of the polymer (phase A), using conventional misers, including (in the preparation of dispersion) vibrators and high-speed stirrers, etc. The dispersion of phase (A), containing the core material in solution or in suspension, is carried out in the aqueous phase (B), again using conventional mixers, such as high-speed mixers, vibration mixers, or even spray nozzles, in which case the particle size of the microgranulates will be determined not only by the concentration of phase (A), but also by the emulsate or microparticulate size. With conventional techniques for the microencapsulation of biological or pharmaceutical agents (active agents), the microparticles form when the solvent containing an active agent and a polymer is emulsified or dispersed in an immiscible solution by stirring, agitating, vibrating, or some other dynamic mixing technique, often for a relatively long period of time.

Conventional methods for the construction of microparticles of the invention are also described in U.S. Pat. No. 4,389,330, and U.S. Pat. No. 4,530,840, both or which are incorporated herein by reference in their entirety. U.S. Pat. No. 4,389,330 describes the following method. The desired agent is dissolved or dispersed in an appropriate solvent, To the agent-containing medium is added the polymeric matrix material in an amount relative to the active ingredient which gives a product of the desired loading of active agent. Optionally, all of the ingredients of the microparticle product can be blended in the solvent medium together. Suitable solvents for the agent and the polymeric matrix material include organic solvents such as acetone, halogenated hydrocarbons such as chloroform, methylene chloride and the like, aromatic hydrocarbon compounds, halogenated aromatic hydrocarbon compounds, cyclic ethers, alcohols, ethyl acetate and the like. Preferred solvents for the agent are methylene chloride or ethyl acetate.

The mixture of ingredients in the solvent can be emulsified in a continuous-phase processing medium; the continuous-phase medium being such that a dispersion of microdroplets containing the indicated ingredients is formed in the continuous-phase medium. Naturally, the continuous-phase processing medium and the organic solvent must be immiscible, and most commonly is water although nonaqueous media such as xylene and toluene and synthetic oils and natural oils can be used. Usually, a surfactant is added to the continuous-phase processing medium to prevent the microparticles from agglomerating and to control the size of the solvent microdroplets in the emulsion. A preferred surfactant-dispersing medium combination is a 1 to 10 wt. % poly (vinyl alcohol) in water mixture. The dispersion is formed by mechanical agitation of the mixed materials. An emulsion can also be formed by adding small drops of the active agent-wall forming material solution to the continuous phase processing medium. The temperature during the formation of the emulsion is not especially critical but can influence the size and quality of the microparticles and the solubility of the drug in the continuous phase. Of course, it is desirable to have as little of the agent in the continuous phase as possible. Moreover, depending on the solvent and continuous-phase processing medium employed, the temperature must not be too low or the solvent and processing medium will solidify or the processing medium will become too viscous for practical purposes, or too high that the processing medium will evaporate, or that the liquid processing medium will not be maintained. Moreover, the temperature of the medium cannot be so high that the stability of the particular agent being incorporated in the microparticles is adversely affected. Accordingly, the dispersion process can be conducted at any temperature which maintain stable operating conditions, which preferred temperature being about 30° C. to 60° C., depending upon the drug and excipient selected.

The dispersion which is formed is a stable emulsion and from this dispersion the organic solvent immiscible fluid can optionally be partially removed in the first step of the solvent removal process. The solvent can easily be removed by common techniques such as heating, the application of a reduced pressure or a combination of both. The temperature employed to evaporate solvent from the microdroplets is not critical, but should not be that high that it degrades the agent employed in the preparation of a given microparticle, nor should it be so high as to evaporate solvent at such a rapid rate to cause defects in the wall forming material. Generally, from 5 to 75%, preferably 1 to 25% of the solvent is removed in the fiast solvent removal step.

After the first stage, the dispersed microparticles in the solvent immiscible fluid medium are isolated from the fluid medium by any convenient means of separation. Thus, for example, the fluid can be decanted from the microparticle or the microparticle suspension can be filtered. Still other, various combinations of separation techniques can be used if desired.

Following the isolation of the microparticles from the continuous-phase processing medium, the remainder of the solvent in the microparticles is removed by extraction. In this step, the microparticles can be suspended in the same continuous-phase processing medium used in step one, with or without surfactant, or in another liquid. The extraction medium removes the solvent from the microparticles and yet does not dissolve the microparticles. During the extraction, the extraction medium with dissolved solvent can optionally be removed and replaced with fresh extraction medium. This is best done on a continual basis. Obviously, the rate of extraction medium replenishment or a given process is a variable which can easily be determined at the time the process is performed and, therefore, no precise limits for the rate must be predetermined. After the majority of the solvent has been removed from the microparticles, the microparticles are dried by exposure to air or by other conventional drying techniques such as vacuum drying, drying over a desiccant or the like. This process is very efficient in encapsulating the agent since core loadings of up to 80 wt. %, preferably up to 60 wt. % are obtained.

Alternatively, and preferably, controlled-release ricropartcles containing an active agent can be prepared through the use of static mixers as described in U.S. Pat. No. 5,733,566, incorporated herein by reference in its entirety. Static or motionless mixers consist of a conduit or tube in which is received a number of static mixing agents. Static mixers provide homogeneous mixing in a relatively short length of conduit, and in a relatively short period of time. With static mixers, the fluid moves through the mixer, rather than some part of the mixer, such as a blade, moving through the fluid.

A static mixer can be used to create an emulsion. When using a static mixer to form an emulsion, several factors determine emulsion particle size, including the density and viscosity of the various solutions or phases to be mixed, volume ratio of the phases, interfacial tension between the phases, static mixer parameters (conduit diameter; length of mixing element; number of mixing elements), and linear velocity through the static mixer. Temperature is a variable because it effects density, viscosity, and interfacial tension. The controlling variables are linear velocity, sheer rate, and pressure drop per unit length of static mixer.

In order to create microparticles containing an active agent, an organic phase and an aqueous phase are combined. The organic and aqueous phases are largely or substantially immiscible, with the aqueous phase constituting the continuous phase of the emulsion. The organic phase includes an active agent as well as a wall-forming polymer or polymeric matrix material. The organic phase can be prepared by dissolving an active agent in an organic or other suitable solvent, or by forming a dispersion or an emulsion containing the active agent. The organic phase and the aqueous phase are pumped so that the two phases flow simultaneously through a static mixer, thereby forming an emulsion which comprises microparticles containing the active agent encapsulated in the polymeric matrix material. The organic and aqueous phases are pumped through the static mixer into a large volume of quench liquid to extract or remove the organic solvent. Organic solvent may be removed from the microparticles while they are washing or being stirred in the quench liquid. After the microparticles are washed in a quench liquid, they are isolated, as through a sieve, and dried.

An alternative process involves the use of a static mixer with co-solvents, This process outlined below, is also described in U.S. Pat. No. 5,733,566 herein incorporated by reference. In this process for preparing biodegradable microparticles comprising a biodegradable polymeric binder and a biologically active agent, a blend of at least two substantially non-toxic solvents, free of halogenated hydrocarbons, is used to dissolve both the agent and the polymer, The solvent blend containing the dissolved agent and polymer is dispersed in an aqueous solution to form droplets. The resulting emulsion is then added to an aqueous extraction medium preferably containing at least one of the solvents of the blend, whereby the rate of extraction of each solvent is controlled, whereupon the biodegradable microparticles containing the biologically active agent are formed. The process has the advantages that less extraction medium is required because the solubility of one solvent in water is substantially independent of the other and solvent selection is increased, especially with solvents that are particularly difficult to extract.

Accordingly, a number of conventional particle formation techniques can be carried out, for example spray drying, extrusion cutting and drying (spherunization), fluid bed coating of seed particles, coacervation, collection and drying, wet granulation and milling, milling of solids or larger particles, precipitation of amorphous solids, fluid bed granulation, evaporation or air drying followed by milling, spray or melt cooling or prilling, spray freeze-drying, compression (i.e., pelleting and grinding) and solution-loading of preformed matrix particles, (i.e., sequential swelling and drying).

The spray freeze-drying process is a viable process in terms of process efficiency (higher yields, narrower particle size distributions), and formulation flexibility. The major disadvantage though is the potential porous morphology associated with the process due to the sublimation of the fine ice crystals leaving pores after freeze drying. It may hinder the efficiency for needleless syringe Applications where dense particles might be required for skirt penetration. Therefore, a formulation and/or a drying condition that can result in shrunk (or collapsed) particles is preferred. Ideally, excipients with Tg's (glass transition temperatures) lower than the primary drying temperature allow particles to collapse and result in dense particles.

Alternatively, hydrogel particles can be loaded with a guest substance, and the localized hydrogel environment manipulated to lower solubility characteristics of the guest, thereby forming a sustained release composition in accordance with the present invention. Hydrogel compositions are well-known in the biomedical arts, and are commonly used as substrates for cell and tissue culture, impression materials for prosthetics, wound-packing materials, or as solid phase materials in size exclusion or affinity chromatography applications. For example, nonporous, deformed and/or derivatized agarose hydrogel compositions have been used in high-performance liquid chromatography and affinity chromatography methods (Li et al (1990) Preparative Biochem 20 107-121), and superporous agarose hydrogel beads have been used as a support in hydrophobic interaction chromatography (Gustavsson et al (1999) J. Chromatography 830 275-284). In the pharmaceutical fields, hydrogel monomers (natural or synthetic) are commonly added to pharmaceutical compositions (with an initiator and, sometimes, crosslinking agents) and then allowed to polymerize, thereby encapsulating a guest pharmaceutical within a hydrogel matrix. These techniques are used to provide microsphere carrier systems for drug targeting or controlled release systems. For example, cross-linked hydrogel microspheres have been used to encapsulate islet cells for the treatment of diabetes (Lim et al (1980) Science 210 908-910) or cancer cells that produce cancer-suppressing materials (U.S. Pat. No. 5,888,497), and biodegradable hydrogel microspheres are widely used to encapsulate a wide variety of drug compositions, most commonly peptides and proteins (Wang et al (1997) Pharm, Dev. and Technology 2, 135-142).

In our commonly owned International Application No. PCT/GB00/00349, we describe the use of pre-formed hydrogel particles, for example, agarose or dextran particles, as carrier systems for drugs, vaccines, diagnostics and other guest substances. These hydrogel particles can be based as carrier systems for a wide variety of pharmacologically active guest agents, and are quite robust, making them uniquely well-suited for high-velocity particle injection delivery techniques. Since release of the guest agent from some such hydrogels will typically be dependent upon several factors, e.g. degree of swelling experienced by the hydrogel when delivered to an aqueous environment; dissolution of the crystallized guest agent; the cross-linking density of the hydrogel matrix; diffusion of the active from the hydrogel matrix; degradation of the hydrogel matrix; the pH and ionic strength of the target site; and the like, numerous delivery profiles can be readily tailored for each guest agent.

To form sustained release hydrogel compositions suitable for use in a transdermal particle delivery system, a suitable hydrogel particle is loaded with a guest substance as described in International Application No. PCT/GB00/00349, and then excess amounts of a counter-ion, typically in the form of a salt, are added to the loaded particles to form an aggregate (e.g. divalent cations bind preferentially with histidine sites on a peptide or protein therapeutic agent, such as human growth hormone, to form a less soluble aggregate). In any event, the counter-ions are used to associate with the guest substances, thereby lowering the available dissolution concentration of the guest as governed by the solubility of the guest/counter ion aggregate product from the hydrogel matrix. In one embodiment, a protein or peptide guest is loaded into a pre-formed hydrogel particle along with one or more suitable excipients, and the resulting loaded particles are dried. A pharmaceutically acceptable salt solution (e.g. sodium chloride) is then drawn into the loaded hydrogel particles to form aggregates with the protein guest, after which the particles are again dried. Upon administration of the particles to a target tissue site, water is absorbed by the hydrated hydrogel structure. An abundance of the dissolving salt within the discrete environment of the hydrogel matrix holds the local concentration of the counter-ion near saturation, thereby lowering the solubility of the guest protein and slowing its release from the hydrogel particle to provide a sustained release delivery profile. The higher the ratio of salt to guest is within the hydrogel particles, the more pronounced the sustained release effect will be.

Additionally or alternatively, complexing agents can be added to such formulations to form less soluble metal/guest (e.g. protein) aggregates within the hydrogel carrier particle, whereby the lowered solubility of the complexed product creates a sustained release composition in accordance with the invention. In one embodiment, a zinc composition (in the form of the chloride or acetate salt) is used in an aggregation technique to control the release of a peptide from a hydrogel carrier particle. The peptide, e.g. human growth hormone, is loaded into a suitable hydrogel particle using the methods of International Application No. PCT/GB00/00349. The protein guest is added to the beads from a solution of about 10-200 mg/ml, together with stabilizing excipients such as trehalose or glycine, and the loaded hydrogel particles are then dried using a suitable drying technique such as lyophilisation. A zinc acetate solution is prepared at near saturation and then adsorbed into the dry hydrogel particles in a second loading step. The particles are then again dried, e.g. by lyophilisation. Zinc-human growth human complexes will then occur upon dissolution of the guest protein during the second loading step, and/or upon rehydration of the particle upon delivery into an aqueous environment, e.g. a target tissue site.

The microparticle composition of this invention has particles of a size appropriate for high velocity delivery to an animal across the stratum corneum or a transmucosal membrane. The mean mass aerodynamic diameter (MMAD) of the particles is preferably larger than about 0.1 microns or about 10 microns, but smaller than about 100 microns, particularly less than 75 microns. Preferably, the majority of the particles will be in the range of about 20 to 75 microns. Typically a substantially homogenous population of particles of approximately the same size is provided. For example, less than 10% by weight of the particles may have a diameter which is 5 microns more or 5 microns less than the 1. For drug delivery, an optimal particle size is usually at least about 10 to 100 microns (larger than the size of a typical cell). For gene delivery, an optimal particle size is generally substantially smaller than 10 micron, e.g. 0.1 to 5 microns.

The average particle size of the powder can be measured as, a mass mean aerodynamic diameter (MMAD) using conventional techniques such as microscopic techniques (where particles are sized directly and individually rather than grouped statistically), absorption of gasses, or permeability techniques. If desired, automatic particle-size counters can be used (eg., the Coulter Counter, MAC Counter, or Gelman Automatic Particle Counter) to ascertain average particle size.

Once formulated, the particles can be assessed using the following methods. Particle characterization for powder injection has different requirements than for traditional pharmaceutical products. Most standard solid dosage forms place the highest value on facile dissolution. For example, in the preparation of tablets, density and particle size or morphology do not play an important role and individual particle density, an important parameter in determining momentum for powder injection, is typically not considered as long as flow characteristics are acceptable. However, for powders for direct supersonic injection, individual particle density is important and thus multiple methods of characterization are typically used. Absolute and theoretical density determinations are made and correlated with delivery, efficiency.

Actual particle density, or “absolute density,” can be readily ascertained using known quantification techniques such as helium pycnometry and the like. Alternatively, envelope (“tap”) density measurements can be used to assess the density of a particulate pharmaceutical composition produced according to the methods of the invention, Envelope density information is particularly useful in characterizing the density of objects of irregular size and shape. Envelope density, or “bulk density,” is the mass of an object divided by its volume, where the volume includes that of its pores and small cavities. Other, indirect methods are available which correlate to density of individual particles. A number of methods of determining envelope density are known in the art including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Particle morphology, particularly the aerodynamic shape of a particle, can be readily assessed using standard light microscopy. It is preferred that the particles have a substantially spherical or at least substantially elliptical aerodynamic shape. It is also preferred that the particles have an axis ratio of 3 or less, i.e. from 3:1 to 1:1, to avoid the presence of rod- or needle-shaped particles. These same microscopic techniques can also be used to assess the particle surface characteristics, e.g., the amount and extent of surface voids or degree of porosity.

Another key attribute, particle size determination, is well known to be significantly influenced by methodology (Etzler, F. M., Sauderson, M. S., Particle size analysis: a comparative study of various methods, Part. Part. Syst. Charact., 12, 217, 1995). The API Aerosizer® (Amherst Process Instruments, Hadley, Mass.) is a supersonic time of flight device in which the mode of measurement closely parallels the energetic helium jet basic to powder injection. Data from this instrument, however, must be augmented with light microscopy including computer-aided image analysis techniques. Such information has also been compared to non-solvent suspension laser diffraction methodology, light obscuration techniques, and Coulter Counter® volume measurements. The latter techniques have been especially powerfull to evaluate particles before and after passage through the transient high-energy acceleration jet and supersonic mode of drug delivery. As described below, metallized film or rigid foam targets give some quantitative information on the powder energy upon injection. Quantitative techniques which are directly related to the powder injection process are measurement of particle size distribution pre vs post passage through a needleless syringe device and particle micro-hardness (i.e., impact strength when injected against a hard surface) (Ghadiri, M., Zhang, Z., Impact attrition of particulate solids, IFPRI Final Report, FRR 16-03 University of Surrey, UK, 1992). Other techniques include direct particle indentation techniques (i.e,, Nano Indenter II®, MTS Systems Corp, Oak Ridge, Tenn. or Micro Hardness Tester®, Anton Paar GmbH, Graz, Austria) in which a probe is used to measure the effect of force against a single particle on a light microscope stage.

Other tests of the properties of a formulation include in vitro skin penetration using full thickness human skin and Franz-type diffusion cells used to measure delivery but also provide indication of drug dissolution and transport. A parallel to the metallized film energy test with a biological target is the use of TEWL (transepidemial water loss) measurements on skin in vitro after delivery. Particles are studied during formulation programs as well as pre and post delivery by light microscopy and scanning electron microscopy (SEM) to determine their initial morphology and/or changes during the transit of the highly energetic supersonic helium flow.

The drug is delivered to the animal by high velocity delivery into skin or mucosal sites using the energy of a transient helium gas jet at a predetermined area of skin or mucosal tissue. A “predetermined area” is intended to be a defined area of intact unbroken living skin or mucosal tissue. That area will usually be in the range of about 0.3 cm² to about 10 cm². However, it will be appreciated by those skilled in the art of high velocity drug delivery that the area of skin or mucosal tissue through which drug is administered may vary significantly, depending on device configuration, dose, and the like.

One particular needleless syringe generally comprises an elongate tubular nozzle having a rupturable membrane initially closing the passage through the nozzle and arranged substantially adjacent to the upstream end of the nozzle. Particles of a therapeutic agent to be delivered are disposed adjacent to the rupturable membrane and are delivered using an energizing means which applies a gaseous pressure to the upstream side of the membrane sufficient to burst the membrane and produce a supersonic gas flow (containing the pharmaceutical particles) through the nozzle for delivery from the downstream end thereof. The particles can this be delivered from the needleless syringe at delivery velocities of between Mach 1 and Mach 8 which are readily obtainable upon the bursting of the rupturable membrane.

A detailed description of the needleless syringe devices useful in the process of this invention is found in tie, prior art. These devices are referred to as needleless syringe devices a are represented by a dermal PowderJect® System and an oral PowderJect® System. Needleless syringe devices for delivering particles are described in U.S. Pat. No. 5,630,796 to Bellhouse et al, incorporated herein by reference. Although a number of specific device configurations are now available, such devices are typically provided as a pen-shaped instrument containing, in linear order moving from top to bottom, a gas cylinder, a particle cassette or package, and a supersonic nozzle with an associated silencer medium. The microparticle composition or powder is sealed within a particle cassette, e.g., a container formed by two rupturable polymer membranes that are heat-sealed to a washer-shaped spacer to form a self-contained sealed unit. The membrane material is selected to achieve a specific mode of opening and burst pressure that dictate the conditions at which the supersonic flow is initiated. In operation, the device is actuated to release the compressed gas from the cylinder into an expansion chamber within the device. The released gas contacts the particle cassette and, when sufficient pressure is built up, suddenly breaches the cassette membranes sweeping or entraining the particles into the supersonic nozzle for subsequent delivery. The nozzle is designed to achieve a specific gas velocity and flow pattern to deliver a quantity of particles to a target surface of predefined area. The silencer is used to attenuate the noise of the “sonic boom” produced by the membrane rupture.

A second needleless syringe device for delivering particles is described in commonly owned International Publication No. WO 96/20022. This delivery system also uses the energy of a compressed gas source to accelerate and deliver powdered compositions; however, it is distinguished from the system of U.S. Pat. No. 5,630,796 in its use of a shock wave instead of gas flow to accelerate the particles. More particularly, an instantaneous pressure rise provided by a shock wave generated behind a flexible dome strikes the back of the dome, causing a sudden eversion of the flexible dome in the direction of a target surface. This sudden eversion catapults a powdered composition (which is located on the outside of the dome) at a sufficient velocity, thus momentum, to penetrate target tissue, e.g., oral mucosal tissue. The powdered composition is released at the point of full dome aversion. The dome also serves to completely contain the high-pressure gas flow which therefore does not come into contact with the tissue. Because the gas is not released during this delivery operation, the system is inherently quiet. This design can be used in other enclosed or otherwise sensitive applications for example, to deliver particles to minimally invasive surgical sites.

Another needleless syringe configuration generally includes the same elements as described above, except that instead of having the pharmaceutical particles entrained within a supersonic gas flow, the downstream end of the nozzle is provided with a bistable diaphragm which is moveable between a resting “inverted” position (in which the diaphragm presents a concavity on the downstream face to contain the pharmaceutical particles) and an alternate “everted” position (in which the diaphragm is outwardly convex on the downstream face as a result of a supersonic shockwave having been applied to the upstream face of the diaphragm). In this manner, the pharmaceutical particles contained within the concavity of the diaphragm are expelled at a high initial velocity from the device for transdermal delivery thereof to a targeted skin or mucosal surface.

The actual distance which the delivered particles will penetrate depends upon particle size (e.g., the nominal particle diameter assuming a roughly spherical particle geometry), particle density, the initial velocity at which the particle impacts the skin surface, and the density and kinematic viscosity of the skin. In this regard, preferred particle densities for use in needleless injection generally range between about 0.1 and 25 g/cm³, and more preferably between about 0.8 and 1.5 g/cm³. The injection velocities preferably range between about 100 and 3,000 m/sec.

In preparing powders according to the invention suitable for needleless delivery, it is important that the individual particles have a particular size range for optimal delivery, as discussed above. This size range preferably is about 10 microns to about 100 microns in effective diameter, preferably no more than 75 microns. It is also important that the individual particles have a structural integrity and density to survive the unique action of the gas jet with the delivery device and the ballistic impact with the skin or mucosal membrane at high velocities. It is further important that the vast majority of the particles making up a unit dosage powder for delivery are within the desired size range and stay within the size range during storage and delivery of the agent. It is further important that the pharmacologically-active agent is stable in the particle during storage and that the excipient present in the particles allow the active agent to be released (by dissolution or diffusion) to act locally or to be transported systemically to the intended site of action.

Single unit dosages or multidose containers, which are adapted for use in a needleless syringe and in which the particles of the invention may be packaged prior to use, can comprise a hermetically sealed container enclosing a suitable amount of the particles that make up a suitable dose. The particle compositions can be packaged as a sterile formulation, and the hermetically sealed container can thus be designed to preserve sterility of the formulation until use in the methods of the invention. If desired, the containers can be adapted for direct use in the above-referenced needleless syringe systems.

Powders of the present invention can thus be packaged in individual unit dosages for delivery via a needleless syringe. As used herein, a “unit dosage” intends a dosage receptacle containing a therapeutically effective amount of a powder of the invention. The dosage receptacle typically fits within a needleless syringe device to allow for transdermal delivery from the device. Such receptacles can be capsules, foil pouches, sachets, cassettes or the like.

The container in which the particles are packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the hydrogel compositions contained therein for human administration.

If desired, needleless syringes can be provided in a preloaded condition containing a suitable dosage of the hydrogel particles described herein. The loaded syringe can be packaged in a hermetically sealed container, which may further be labeled as described above.

A number of test methods have been developed, or established test methods modified, in order to characterize performance of needleless syringe devices and the microparticle composition of this invention. These tests range from characterization of the powdered composition, assessment of the gas flow and particle acceleration, impact on artificial or biological targets, and measures of complete system performance.

Assessment of Effect on Artificial Film Targets

A functional test that measures many aspects of powder injection systems simultaneously has been designated as the “metallized film” or “penetration energy” (PE) test. It is based upon the quantitative assessment of the damage that particles can do to a precision thin metal layer supported by a plastic film substrate. Damage correlates to the kinetic energy and certain other characteristics of the particles. The higher the response from the test (i.e., the higher the film damage/disruption) the more energy the device has imparted to the particles. Either electrical resistance change measurement or imaging densitometry, in reflectance or transmission mode, provide a reliable method to assess device or formulation performance in a controllable and reproducible test.

The film test-bed has been shown to be sensitive to particle delivery variations of all major device parameters including pressure, dose, particle size distribution and material, etc. and to be insensitive to the gas. Aluminum of approximately 350 Angstrom thickness on a 125 μm polyester support is currently used to test devices operated at up to 60 bar.

The metallized film test is extremely useful for device and formulation development purposes and also holds promise as a quality control tool. If all parameters except one are held constant it can be used as a relative measure to compare nozzles or drug cassette designs or formulations. The test can give three types of information about system performance; total energy as well as spatial and temporal energy distribution. One analysis mode generates a single measure of impact across the entire target area. Alternative analysis give a spatially resolved indication of the homogeneity of delivery across the target. If used in a mode of electrical resistance measurement the test can provide temporal information on particle impact.

Assessment of Impact Effect on Engineering Foam Targets

Another means of assessing relative delivery performance of a needleless syringe device is to gauge the effect of impact on a rigid polymethylimide foam (Rohacell 5 IIG, density 52 kg/m³, Rohm Tech Inc., Malden, Mass.). The experimental set-up for this test is similar to that used in the metallized film test. The depth of penetration is measured using precision calipers. For each experiment a processed mannitol standard is run as comparison and all other parameters such as device pressure, particle size range, etc., are held constant. Data also show this method to be sensitive to differences in particle size and pressure. Processed mannitol standard as an excipient for drugs has been proven to deliver systemic concentrations in preclinical experiments, so the relative performance measure in the foam penetration test has a practical in vivo foundation. Promising microparticle powders can be expected to show equivalent or better penetration to mannitol for anticipation of adequate performance in preclinical or clinical studies. This simple, rapid test has value as a relative method of evaluation of powders and is not intended to be considered in isolation.

Particle Attrition Test

A further indicator of particle performance is to test the ability of various candidate compositions to withstand the forces associated with high-velocity particle injection techniques, that is, the forces from contacting particles at rest with a sudden, high velocity gas flow, the forces resulting from particle-to-particle impact as the powder travels through the needleless syringe, and the forces resulting from particle-to-device collisions also as the powder travels through the device. Accordingly, a simple particle attrition test has been devised which measures the change in particle size distribution between the initial composition, and the composition after having been delivered from a needleless syringe device.

The test is conducted by loading a particle composition into a needleless syringe as described above, and then discharging the device into a flask containing a carrier fluid in which the particular composition is not soluble (e.g., mineral oil, silicone oil, etc.). The carrier fluid is then collected, and particle size distribution in both the initial composition and the discharged composition is calculated using a suitable particle sizing apparatus, e.g., an AccuSizer® model 780 Optical Particle Sizer. Compositions that demonstrate less than about 50%, more preferably less than about 25% or less than about 20%, reduction in mass mean diameter (as determined by the AccuSizer apparatus) after device actuation are deemed suitable for use in the needleless syringe systems described herein.

Deliver to Human Skin In Vitro and Transepidermal Water Loss

For a preclinical system performance test that more closely parallels eventual practical use, microparticles or model particles are injected into dermatomed, full thickness human abdomen skin samples. Replicate skin samples after injection can be placed on modified Franz diffusion cells containing 32° C. water, physiologic saline or buffer. Additives such as surfactants may be used to prevent binding to diffusion cell components. Two kinds of measurements can be made to assess either physical attributes of the delivery or performance of the formulation in the skin.

To measure physical effects, i.e. the effect of powder injection on the barrier function of skin, the transepidermal water loss (TEWL) can be measured. Measurement is performed at equilibrium (approximately 1 hour) using a Tewameter TM 210® (Courage & Khazaka, Koln, Ger) placed on the top of the diffusion cell cap that acts like an approximately 12 mm chimney. Larger particles and higher injection pressures generate proportionally higher TEWL values in vitro and this has been shown to correlate with results In vivo (Kwon, S. Y., Burkoth, T. L., Transdermal powder injection effect on trans-epidermal water loss (TEWL) and drug delivery, Pharm. Sci. suppl.1 (1), 103, 1998). Upon powder injection in vitro TEWL values increased from approximately 7 to approximately 27 (g/m²h) depending on particle size and helium gas pressure. Helium injection without powder has no effect. In vivo, the skin barrier properties return rapidly to normal as indicated by the TEWL returning to pretreatment values in approximately 1 hour for most powder sizes. For the largest particles, 53-75 μm, skin samples show 50% recovery in an hour and full recovery by 24 hours.

Delivery to Human Skin In Vitro and Drug Diffusion Rate

To measure the formulation performance In vitro, drug can be collected by complete or aliquot replacement of the Franz cell receiver solution at predetermined time intervals for chemical assay using HPLC or other suitable analytical technique. Concentration data can be used to generate a delivery profile and calculate a steady state permeation rate. In vitro, the amount of drug delivered through the skin over 24 hours increased proportionally with the TEWL value (R²=0.78). This technique can be used to screen formulations for early indication of drug binding to skin, drug dissolution, efficiency of particle penetration of stratum corneum, etc., prior to in vivo studies.

Experimental

Below is an example of a specific embodiment for carrying out the methods of the present invention. The example is offered for illustrative purposes only, and are not intended to limit the scope of the present invention in any way.

Efforts have been made to ensure accuracy with respect to numbers used (e.g. amounts, temperatures, etc.), but some experimental error and deviation should, of course, be allowed for.

### EXAMPLE

The following study was carried out to evaluate the suitability of using derivatized hydrogel particles to produce a sustained release composition suitable for high velocity transdermal particle delivery according to the methods of the invention.

A preparation of cross-linked, sulfated dexan affinity chromatography resin beads were obtained from a commercial source (Sigma. St, Louis, Mo.). More particularly, 4% cross-linked suflated beaded dextran was obtained as a lyophilized powder stabilized in lactose. The sulfate ligand will bind with a charged guest substance (e.g. a peptide or protein) at low salt concentration. This binding is reversible and the bound guest can be desorbed at a high salt concentration.

In the study, lysozyme (Sigma) was loaded into the beads using the techniques described in commonly held International Application No. PCT/GB00/00349.

Particularly, each gram of the sulfated dextran beads was washed with approximately 3×500 ml deionized water. Excess water was removed by vacuum filtration. A loading solution was prepared by dissolving 4.00 g lysozyme in 200 ml deionized water, yielding a 20 mg/ml solution. The loading solution was added to the hydrated dextran beads at a 1:1 ratio to provide a suspension. The suspension was stirred for one hour, and excess solution was drained from the hydrated beads. The loaded beads were then divided into two portions. The first portion (5.7 cc, experimental) was placed into a column (Econo column, BioRad, Hercules, Calif.) and washed with a phosphate buffered saline (PBS) solution (pH 7.4) for 19.6 hours, then eluted with 21.2 ml of a high salt elution buffer (1.0M ammonium sulfate, 50 mM sodium phosphate, pH 7.2). The second portion (4.7 cc, control) was loaded into a similar column and immediately eluted with the elution buffer alone. The amount of lysozyme released from the dextran beads during the PBS wash was determined by comparison against the amount of lysozyme eluted by the elution solution for both the experimental and the control portions.

Lysozyme concentration was determined using a UV detector (BioRad UV detector). A standard curve (UV absorption vs lysozyme concentration) was prepared by measuring the UV absorption of stock lysozyme solutions at different concentration ranging from 1 mg/ml to 4 mg/ml, Concentrations of the unknowns were then determined by extrapolation of the standard curve. The amount of lysozyme released during the PBS wash of the experimental portion was not detectable with the UV detector. However, the amount of lysozyine eluted in the elution buffer for the experimental (PBS washed beads) and the control portions were 3.72 mg/ml beads and 10.5 mg/ml wet beads, respectively. This indicates that the average sustained release rate of the lysozyme from the hydrogel particles over the 19.6 hour period was 0.3 mg/hr/g wet bead, or 3.2% per hour, where release was substantially linear with time.

### EXAMPLE

The sustained release study described in the above Example was repeated except, in this instance, a different elution rate was used to determine if the rate of release of the lysozyme guest substance from the dextran is a function of the flow rate of the elution buffer.

More particularly, sulfated dextran beads were loaded with lysozyme as described in the above Example. The specifics of the loading were as follows: 42.4 g of the hydrated dextran beads was stirred in 100 ml of lysozyme solution (to yield a 20.11 mg/ml solution) for 1 hour. The loaded beads were then loaded into a column as described above for a further sustained release study.

For the PBS elution, the PBS wash (pH 7.4) was used to elute lysozyme from the beads at a flow rate of 0.84 ml/min for 20.6 hours. The eluted solution was collected in fractions, and the elution fractions were analyzed with HPLC for lysozyme concentration using a Vydac C18 HPLC column. For the high-salt elution, the remaining lysozyme in the beads was then eluted with the high-salt elution buffer at a rate of 0.84 m/min.

The amount of lysozyme protein that eluted with the PBS wash was 39.51 mg and, from the high salt elution, the amount of lysozyme was 40.34 mg. Accumulated percent lysozyme release (Y) was plotted against elution time (X). The resulting sustained release rate curve was found to be linear, and the line equation was determined to be Y=1.49X+0.242, or in other words, the release rate was found to be 1.49%/hr. This indicates that the slope (the sustained release rate) is a function of the flow rate of the elution buffer.

All publications, patents and patent applications are herein incorporated by reference in their entirety to the same extent as if each individual publication, patent or patent application was specifically and individually indicated to be incorporated by reference in its entirety.

As can be appreciated from the disclosure above, the present invention has a wide variety of applications. The invention is intended to cover various modifications and equivalent arrangements included within the spirit and scope of the appended Claims.

1. A particulate composition suitable for administration to a subject by means of a needleless syringe, said composition comprising particles that comprise a biologically active agent and a sustained-release material which controls release of the active agent to the subject following administration of the composition thereto, wherein said particles have a mean mass aerodynamic diameter of from about 0.1 to about 250 microns and an envelope density of from about 0.1 to about 25 g/cm³.

2. The composition of claim 1 wherein the mean mass aerodynamic diameter of the particles is from about 10 to about 100 microns, less than about 10% by weight of the particles have a diameter which is 5 microns more or 5 microns less that the mean mass aerodynamic diameter, the particles have an axis ratio of from about 3:1 to 1:1, the envelope density of the particles is from about 0.8 to about 1.5 g/cm³, and the particles exhibit less than about 25% reduction in mean mass diameter after delivery from a needleless syringe as determined in a particle attrition test.

3. The composition of claim 1 wherein the particles have a mean mass aerodynamic diameter of from about 20 to about 75 microns.

4. The composition of claim 1 wherein the biologically active agent is selected from the group consisting of drugs, vaccines, oligosaccharides, peptides, proteins and nucleic acids.

5. The composition of claim 1 wherein the sustained-release material is selected from the group consisting of poly(lactide), poly(glycolide), poly(carprolactone), poly(hydroxybutyrate), poly(lactide-co-glycolide) and poly(lactide-co-caprolactone).

6. A composition according to claim 1 which comprises a first set of particles comprising the biologically active agent in association with a first sustained-release material and a second set of particles comprising the biologically active agent in association with a second sustained-release material.

7. The composition of claim 1 wherein the particles are microcapsules comprising the biologically active agent encapsulated by a wall-forming sustained-release polymer material.

8. The composition of claim 1 wherein the particles are microspheres comprising a sustained-release polymer material.

9. A hermetically sealed single unit dosage or multidose container adapted for use in a needleless syringe, said container comprising the composition of claim 1.

10. A needleless syringe containing the composition of claim 1.

11. A method for delivering a biologically active agent to a subject, said method comprising:
(a) providing particles which have a mean mass aerodynamic diameter of from about 0.1 to about 250 microns and an envelope density of from about 0.1 to about 25 g/cm³ and which comprise the biologically active agent in association with a sustained-release material which controls release of the active agent to the subject following delivery thereto;
(b) accelerating the particles to a velocity from about 100 to about 3000 m/sec; and
(c) impacting the particles onto a surface of the subject thereby causing the particles to penetrate the surface and enter the subject.

12. The method of claim 11 wherein the particles wherein the mean mass aerodynamic diameter of the particles is from about 10 to about 100 microns, less than about 10% by weight of the particles have a diameter which is 5 microns more or 5 microns less that the mean mass aerodynamic diameter, the particles have an axis ratio of from about 3:1 to 1:1, the envelope density of the particles is from about 0.8 to about 1.5 g/cm³, and the particles exhibit less than about 25% reduction in mean mass diameter after delivery from a needleless syringe as determined in a particle attrition test.

13. The method of claim 11 wherein the particles have a mean mass aerodynamic diameter of from about 20 to about 75 microns.

14. The method of claim 11 wherein the biologically active agent is selected from the group consisting of drugs, vaccines, oligosaccharides, peptides, proteins and nucleic acids.

15. The method of claim 11 wherein the sustained-release material is selected from the group consisting of poly(lactide), poly(glycolide), poly(carprolactone), poly(hydroxybutyrate), poly(lactide-co-glycolide) and poly(lactide-co-caprolactone).

16. The method of claim 11 wherein the composition comprises a first set of particles comprising the biologically active agent in association with a first sustained-release material and a second set of particles comprising the biologically active agent in association with a second sustained-release material.

17. The method of claim 1 1 wherein the particles are microcapsules comprising the biologically active agent encapsulated by a wall-forming sustained-release polymer material.

18. The method of claim 11 wherein the particles are microspheres comprising a sustained-release polymer material.

19. The method of claim 11 wherein the particles are administered to the subject at a speed of 200 meters/second or greater.

20. The method of claim 11 wherein the biologically active agent comprises 1 to 99 weight percent of the particle.

21. The method of claim 11 wherein the particles are administered to a skin of the subject.

22. The method of claim 11 wherein the particles are administered to a tissue of the subject.

23. The method of claim 11 wherein the particles comprise two or more biologically active agents.

24. The method of claim 11 wherein the subject is an animal.

25. The method of claim 11 wherein acceleration of the particles is effected by entraining the particles into a flow of moving gas.

26. The method of claim 11 wherein acceleration of the particles is effected by displacing the particles with a shock wave.
